(19) United States
(12) Patent Application Publication
(10) Pub. No.: US 2024/0293677 A1
(43) Pub. Date: Sep. 5, 2024

(54) SYSTEMS AND METHODS FOR CONTROLLING A MEDICAL DEVICE USING BAYESIAN PREFERENCE MODEL BASED OPTIMIZATION AND VALIDATION

(71) Applicant: Regents of the University of Minnesota, Minneapolis, MN (US)

(72) Inventors: Theoden I. Netoff, Minneapolis, MN (US); David Darrow, Minneapolis, MN (US); Zixi Zhao, Minneapolis, MN (US); Andrew Lamperski, Minneapolis, MN (US)

(73) Assignee: Regents of the University of Minnesota, Minneapolis, MN (US)

(21) Appl. No.: 18/579,222

(22) PCT Filed: Jul. 15, 2022
(86) PCT No.: PCT/US2022/073787

§ 371 Date: Jan. 12, 2024

Related U.S. Application Data
(60) Provisional application No. 63/203,285, filed on Jul. 15, 2021.

(51) Int. Cl. A61N 1/36; A61N 1/372; G16H 20/40; G16H 40/63

(52) U.S. Cl. CPC A61N 1/36139; A61N 1/36062; A61N 1/36171; A61N 1/36175; A61N 1/37264; G16H 20/40; G16H 40/63

(57) ABSTRACT

A medical device is controlled based in part on a Bayesian preference learning-based optimization of the control parameters of the device. The Bayesian preference learning-based optimization is implemented to identify personalized optimal control parameters based on user preference for control parameter settings. The Bayesian preference learning-based optimization provides automatic tuning of the control parameters of the medical device based on feedback data, such as user response data, to achieve a user-specific therapy or effect.

### BACKGROUND

Epidural spinal cord stimulation has been reported to partially restore volitional movement and autonomic functions after motor and sensory-complete spinal cord injury (SCI). Modern spinal cord stimulation platforms offer significant flexibility in spatial and temporal parameters of stimulation delivered. Heterogeneity in SCI and injury-related symptoms necessitate stimulation personalization to maximally restore functions. However, the large multi-dimensional stimulation space makes exhaustive tests impossible.

Tuning spinal cord stimulation device for spinal cord injury is challenging due to the existence of multiple outcomes and a vast parameter space. For example, a clinician has to determine parameters such as pulse frequency and pulse width for a stimulation setting. The stimulation treatment for SCI has been tried with intensive in-patient rehabilitation and measurement instrumentation for finding the optimal settings, which can cause expensive cost to healthcare and the patient. Also, there has not been comprehensive validation methods to properly prove the multi-facet outcomes from optimal settings.

### SUMMARY OF THE DISCLOSURE

The present disclosure addresses the aforementioned drawbacks by providing a controller for controlling a controllable medical device. The controller includes an input that receives feedback data representative of a treatment response or effect in a subject, a processor in communication with the input, a memory in communication with the input and the processor, and an output. The processor is programmed to receive the feedback data from the input and generate a Bayesian preference model therefrom: generate control parameter settings by sampling the Bayesian preference model; and arrange the control parameter settings in an ordered sequence that maximizes information obtained from pairwise comparison of control parameter settings. The memory stores instructions for generating control parameter settings, the feedback data received from the input, and the ordered sequence of control parameter settings generated by the processor. The controller then communicates the ordered sequence of control parameter settings to a controllable medical device (e.g., via the output).

In some aspects, the processor is programmed to arrange the control parameter settings in the ordered sequence such that exploitation of known control parameter settings is maximized and regret in exploration of unknown control parameter settings is minimized. In some other aspects, the processor is programmed to validate the Bayesian preference model according to a validation protocol by at least one of predicting subject preference outcomes of a sequence of comparisons using the Bayesian preference model, or programming the controller with the ordered sequence of control parameter settings and comparing the predicted outcome to a subject preference outcome. Advantageously, the processor can be configured to both arrange the control parameter settings in the ordered sequence such that exploitation of known control parameter settings is maximized and regret in exploration of unknown control parameter settings is minimized and to validate the Bayesian preference model according to a validation protocol by at least one of predicting subject preference outcomes of a sequence of comparisons using the Bayesian preference model, or programming the controller with the ordered sequence of control parameter settings and comparing the predicted outcome to a subject preference outcome.

It is another aspect of the present disclosure to provide a controller for controlling a controllable medical device. The controller includes an input configured to receive feedback data representative of a treatment response or effect in a subject, a memory, a processor in communication with the input and the memory, and an output that communicates the ordered sequence of control parameter settings to a controllable medical device. The memory stores the feedback data received from the input and control parameter settings for controlling a controllable medical device. The processor is programmed to: receive the feedback data from the input: receive control parameter settings from the memory; and arrange the control parameter settings in an ordered sequence for testing the control parameter settings by the subject based at least in part on the feedback data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart of an example method for generating medical device control parameter settings using a Bayesian preference model optimization.

FIG. 2 is an example of a one-dimensional Bayesian optimization plot with relational comparison data. The black dashed line is the fitted preference value function, the blue region is the confidence bound, and the vertical dashed line indicates the next sampling point as determined by the upper confidence bound of the fit model.

FIG. 3 is a flowchart of an example method for generating medical device control parameter settings using a Bayesian preference model optimization, in which control parameter settings are arranged in an ordered sequence by maximizing exploitation and minimizing regret of exploration.

FIG. 4 is a flowchart of an example method for generating medical device control parameter settings using a Bayesian preference model optimization and validation.

FIG. 5 is a flowchart of an example method for generating medical device control parameter settings using a Bayesian preference model optimization and validation, and in which control parameter settings are arranged in an ordered sequence by maximizing exploitation and minimizing regret of exploration.

FIG. 6 is an example workflow diagram for implementing a method for optimizing control parameter settings.

FIG. 7 is a block diagram of an example controller that can control the operation of a medical device based on control parameter settings.

FIG. 8 is a block diagram of an example system for generating control parameter settings using Bayesian preference model optimization techniques described in the present disclosure.

FIG. 9 is a block diagram of example components that can implement the system of FIG. 8.

### DETAILED DESCRIPTION

Described here are systems and methods for controlling a medical device based in part on a Bayesian preference learning-based optimization of the control parameters of the device. The Bayesian preference learning-based optimization is implemented to identify personalized optimal control parameters based on a user's (e.g., a patient's) expressed preference for control parameter settings. The Bayesian preference learning-based optimization provides automatic tuning of the control parameters of the medical device based on feedback data, such as user response data, to achieve a user-specific therapy or effect.

Examples of medical devices that can be controlled in the manner described in the present disclosure include electrical stimulation devices, such as peripheral nerve stimulators, central nervous system stimulators, cardiac pacemakers, and cardiac resynchronization therapy (“CRT”) devices: drug pumps: hearing aids: cochlear implants; and other such devices with controllable parameters.

Each medical device—or in the example of an electrical stimulation device (e.g., a peripheral nerve stimulator, central nervous system stimulator, a cardiac pacemaker, a CRT device), each stimulator—can be programmed with multiple settings, which may then be reprogrammable (e.g., in-clinic with a clinician programmer, or remotely). A batch recommendation and sequencing algorithm can be implemented, in which multiple optimal settings recommendations can be generated and arranged in a testing order that maximizes information gain and minimizes testing cost. The control parameters (e.g., stimulation programs) recommended by the algorithm are programmed on the patient's medical device (e.g., electrical stimulator), and the patient's self-reporting of at-home evaluations are used in preparation of setting recommendations. The patient is instructed to test settings in the order produced by the sequencing algorithm.

In some embodiments, the systems and methods described in the present disclosure implement a Bayesian optimization-based preference learning algorithm to optimize medical device control parameter settings (e.g., the frequency and pulse width of electrical stimulation) based on an individual participant's at-home evaluation, which as one example can be conducted through online preference surveys. The algorithm constructs participant-specific preference models that are then used to suggest new therapeutic settings that efficiently explore the broad parameter space and also focus in on optimal parameter regions. To validate the efficacy of the algorithm, resulting preference model quality can be evaluated through internal and prospective validation.

A patient's preference may not be static and may slowly migrate over time. Preferences might not be static for several reasons, one being that the patient's objective may change over time. For example, their initial preference may focus on the amplitude of movement and then later shift to the control of movement or other more subtle benefits that may align better with improvements in quality of life (e.g., the suppression of spasticity and comfort). The systems and methods described in the present disclosure enable routine updating of control reflected parameter settings based on user preference, thereby allowing for these shifts to be in the updated control parameters.

For many neuromodulation therapies, a device is implanted that can deliver electrical pulses to the brain or spinal cord to reduce symptoms, such as deep brain stimulation for treatment of Parkinson's disease, deep brain stimulation for epilepsy, or spinal cord stimulation for treatment of pain or paraplegia caused by spinal cord injury. These devices can deliver waveforms with different amplitudes, frequencies, pulse widths, and with different spatial patterns, depending on which electrodes are selected to deliver the stimulus. The combination of these different stimulation parameters can result in billions of possible settings, making discovery of an optimal set of parameters that meets a patient's needs challenging. It is an aspect of the present disclosure to provide systems and methods for sequencing a set of control parameter settings into an ordered sequence for testing by the user.

As an example, a sequencing algorithm can be used for selecting a sequence of settings to be evaluated by a user for identifying those settings with high value with minimum regret. By testing two settings and having the user compare and evaluate the one they prefer, it is possible to make models of preference maps. However, when selecting settings to test, the sequence of settings can be important in evaluations. A goal of designing a sequence is to minimize regret. Regret is caused by sampling settings that do not give information, or that are not near the maximum. There is a tradeoff between exploring new settings so as not to be trapped in a local minimum, but also to exploit settings that are known to work for the user.

To address this, the systems and methods described in the present disclosure also provide different sequencing algorithms. As one example, a sequencing algorithm can minimize repeated settings, assuming all settings are of equal value. This is often true when selecting settings for the first time. As another example, a sequencing algorithm can be used when there are high value settings that have been tested, and further testing can refine between them, and exploratory settings, whose values are unknown.

As noted above, the algorithm for medical device control parameter (e.g., electrical stimulation settings) optimization described in the present disclosure implements a Bayesian optimization framework. In general, the optimization goal is to maximize a patient's preference level by finding the optimal control parameters (e.g., temporal stimulation parameters such as frequency and pulse width). The optimization algorithm includes at least three parts. The first part fits a preference model to cumulatively collected data. As an example, the preference model can be implemented as a Gaussian process. The second part, called sampling, determines a new set of settings based on the model. The third part, called sequencing, determines the sequence of settings to be used over a duration of time before the settings are reevaluated. As a non-limiting example, the duration of time may be one month, and a new setting may be tested each data within that duration of time (e.g., within a one-month duration). The sequence effectively determines which two settings are going to be directly compared against each other within a discrete test period (e.g., one day—that is, one setting may be tested on a given day and compared to the setting tested on the previous day).

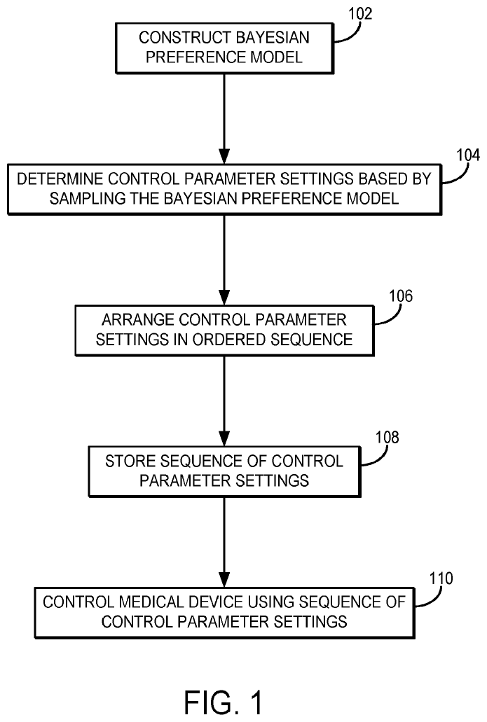

Referring now to FIG. 1A, a flowchart is illustrated as setting forth the steps of an example method for determining a sequence of control parameter settings using a Bayesian preference learning-based optimization.

The method includes constructing a Bayesian preference model, as indicated at step 102. Preference modeling is performed to find a best-fit relationship between a latent variable representing a participant's unobserved preference level and options through Bayes' rule. The best-fit preference model can be the posterior distribution computed through Bayes' rule and the predictive distribution of preference values for unobserved options. As an example, the data set used to extract the model can be composed of binary comparisons.

As a non-limiting example, the systems and methods described in the present disclosure utilize probit modeling with Gaussian process-modeled latent variable to describe preferential, binary observations (i.e., the likelihood in Bayes' rule). Probit modeling enables probability expressions of binary outcomes to be computed or otherwise derived. The Gaussian process-modeled latent variable also dictates the prior distribution expression in Bayes' rule. The Gaussian process property is inherited in the posterior distribution with some approximation, which subsequently enables the derivation of a predictive distribution.

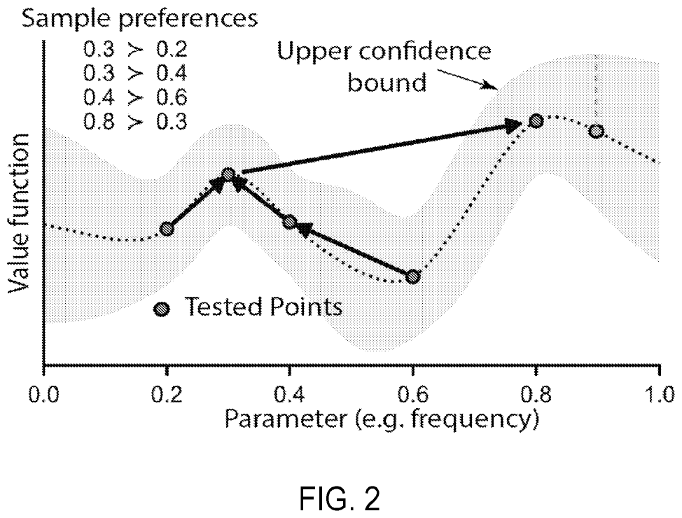

FIG. 2 shows an example of a preference model built based on four binary comparisons. The red dots connected by arrows represent comparisons that have been evaluated, and the arrows point from the losing setting to the winning setting. The dashed line represents the mean of the fitted Gaussian process preference variable, and the blue shaded area represents the confidence bound.

After the preference model is developed, control parameter settings are determined by sampling the Bayesian preference model, as indicated at step 104. For instance, a sampling algorithm is implemented to choose new queries in the parameters to perform the next test. The new queries can be structured or otherwise selected to strike a balance between the exploration of untested parameter regions and the exploitation of tested parameter regions that are known to give desirable results. This balance can be dictated by an acquisition function.

As one example, the acquisition function can be an upper confidence bound (“UCB”) acquisition function. The UCB acquisition function is a weighted sum of the mean and standard deviation predicted by the fitted preference model. For the example in FIG. 2, the weights in the UCB acquisition function are both one, and the UCB is represented by the top edge of the blue shaded area in FIG. 2. The green dot represents the next sample point, as the UCB is the highest at that location.

Often, a medical device is capable of storing multiple different control parameter settings, allowing the patient to choose from a set of selected settings at home for resting on short time durations, such as daily or weekly. Upon clinical visits, multiple different parameter settings can be determined and the medical device can be updated with these new settings for evaluations. The updates of these settings for evaluation may occur on a longer period of time, such as monthly or quarterly. Therefore, the sampling algorithm can be implemented to determine a batch of new parameters to sample instead of a single parameter.

As a non-limiting example, a batch sampling algorithm can be utilized to sequentially select the top q parameter locations from the candidate pool, where q is the batch size. To select each sample, the batch sampling algorithm first simulates the outcomes of comparing of each setting in the pool against the incumbent, which is the tested setting that has the highest UCB when using the UCB acquisition function. The batch sampling algorithm then refits a model considering both the observed data and the simulated data. The parameter that produces a model with the highest sum UCB (S-UCB) is included in the batch and deleted from the candidate pool. The pseudo-code of an example batch sampling algorithm is shown below in Table 1.

Pseudo-code of the batch sampling algorithm

```
Inputs: batch size: q; UCBPower acquisition function parameters: α, β;
preferential data set Dₜ; tested stimulation parameters: Xₜ = {x_{1:n}};
candidates of test parameters to be included in the batch; Xₜₑₛₜ
Initialization: D = Dₜ; X = Xₜ; GP = p(fₜ|Dₜ, Xₜ); fₜ = [f(x₁), f(x₂), . . .,
f(xₙ)]^{T}
Sampling:
  For k = 1:q,
    $Based on GP , find {x}^{+} = arg {max}_{x ∈ X} UCB Power ( x , α , β )$
    For \hat{x} ∈ Xₜₑₛₜ,
      Predict \hat{d} = Pref(E(f(x⁺)), E(f(\hat{x})))
      Fit [symbol]     = p(\hat{f}|D ∪ {\hat{d}}, X ∪ {\hat{x}})
      Calculate S-UCBPower(\hat{x}) =Σ_{x∈Xt∪(x)} UCBPower(x, α, β)
    Obtain the kth point, xₖ, for the batch:
    ${x}_{k} = arg {max}_{\hat{x} ∈ {X}_{t e s t}} S - UCB Power ( x , α , β )$
    Augment D = D ∪ {Pref(E(f(x⁺)), E(f(xₖ)))}
    Augment X = X ∪ {xₖ}
    Update GP = p(f|D, X), f = [f(x₁), f(x₂), . . ., f (xₙ)]^{T}
    Update Xₜₑₛₜ = Xₜₑₛₜ\{xₖ}
```

After control parameter settings have been determined, they are arranged in an ordered sequence, as indicated at step 106. As one example, the control parameter settings can be arranged in an ordered sequence that maximizes the information obtained from pairwise comparison of parameter settings. In some other examples, arranging the control parameter settings in the ordered sequence can include selecting a sequence of settings to be evaluated by the user for identifying those settings with high value with minimum regret. Three non-limiting examples of sequencing are described.

One example of sequencing is to randomly select a setting from the programmed settings. If the selected setting is a repeat of the previous setting, then a new setting can be randomly selected by resampling. Advantageously, this sequencing algorithm can be used to measure patient/user preference with direct measures and not through comparisons.

Another example for sequencing is to select a sequence of settings that avoids self-comparisons and repeated comparisons. For instance, if the sequence comparing setting 1 to setting 2 is tested, then the algorithm will avoid testing setting 1 against setting 2 again, or testing setting 2 against setting 1.

An N×N table, array, or matrix can be generated, where N is the number of settings to be tested. The rows in the table represent the setting on one day (e.g., the current setting) and the columns in the table represent the settings on the subsequent day (e.g., the next setting). The settings on the diagonal of the table are set with a value to indicate that these entries should not be used, since they would represent comparing a given setting to itself (e.g., having the same settings two days in a row). As an example, the diagonal entries can be set with values of −1, an “x”, or so on.

The other entries in the table are initially empty. One of the empty entries can be selected, which then defines the initial pair of settings (i.e., the setting for the current day and the setting for the next day), and this entry can be filled in. Any non-zero value in the matrix will suffice to show that it has been used (preferably a different value than those used for the diagonal entries). The column number of the first selection becomes the row of the next selection, and an empty setting is selected from the available settings. After each setting, the reciprocal/mirror entry is filled, so that the same settings being compared in reverse order is avoided. For example, if the first entry selected is [1,3], then a value is entered at that entry and at [3,1].

The next point to test is then selected. In the example above, the first setting that was selected was setting 1, and the next setting was setting 3 (i.e., entry [1,3] was selected first). The next day, setting 3 will be the current setting, so any open entry at random can be selected from row 3 to select the next point. For example, the second point in the sequence can be selected as [3,4]. As noted above, a value can be entered for that entry and its mirror entry, [4,3]. This process is repeated until all entries in the table are filled. If a row is filled, then one of the empty entries can be selected at random and the process can be continued with that pair in the sequence.

An example sequence table, or array or matrix, that can be generated in this manner is illustrated in Table 2 below, and pseudo-code for the sequencing algorithm is shown in Table 3. In this example, the ordered sequence of control parameter settings was {1,4,3,1,5,3,2,5,4,2,1}, which was generated to minimize repeated comparisons.

| | next | | | | |
|---|---|---|---|---|---|
| Current | 1 | 2 | 3 | 4 | 5 |
| 1 | x | 10 | 3 | 1 | 4 |
| 2 | 10 | x | 6 | 9 | 7 |
| 3 | 3 | 6 | x | 2 | 5 |
| 4 | 1 | 9 | 2 | x | 8 |
| 5 | 4 | 7 | 5 | 8 | x |

Pseudo-code of the second sequencing algorithm

```
Inputs: number of settings: k; Sequence length: N;
Outputs: Sequence of settings S, where the value, Sₖ, represents the setting to be tested on
day k.
Initialization: A matrix M is generated to keep track of setting comparisons that have been
    tested. M is initialized so that M = − [symbol]   ^{kxk}, where [symbol]   ^{kxk} is the identity matrix of size kxk;
    The first setting is chosen by selecting a row at random, r₁.
Sampling:
for k = 1: N−1
  Select Cᵢ[rᵢ, cᵢ]:
      if (i > 1) The row, representing the current setting, is determined by the next setting
          from the previous selection, rᵢ = cᵢ₋₁;
      The next setting, cᵢ, is selected at random from the cells within row rᵢ whose values
        are zero.
      If there are no zero values in the row, then a random cell with zero value in the array
        and rᵢ andcᵢ will be this selection.
        Set cell Cᵢ = i
      If the array is full, then initialize the matrix and repeat until N samples in the
        sequence have been generated taking care not to repeat the last sample to the first
        sample after reinitializing.
    Add row to the sample Sᵢ = rᵢ
S_{N} = c_{N-1}
```

In the previous sequencing algorithms, all settings were treated as having equal value. In the third sequencing algorithm settings can be divided into two categories: exploratory settings (so called because they haven't been tested yet) and exploitatory settings (so called because they have been tested and are known to be favorites of the user, or to otherwise have high values). The sequencing algorithm can be configured to maximize exploitation and minimize regret in exploration.

For instance, the sequencing algorithm can sample more times between the known settings, allowing repeated comparisons but in reverse order, and will allow comparison of unknown settings only to known settings. Comparing two unknown settings has little value, because they are not well characterized. This sequencing algorithm can be referred to as a “hub-and-spoke” selection, with the known settings making the hub and sampling exploratory settings and returning the hub making the spokes.

In general, this hub-and-spoke sequencing algorithm can proceed as follows. First, an N×N table, array, or matrix of control parameter settings is generated. The table can be structured so that the exploitatory settings are the first settings and the exploratory are the subsequent settings. The table is divided into four quadrants: exploitatory only, exploratory only, and two that are a combination of exploitatory and exploratory. All of the entries along the diagonal can be filled or otherwise blocked to prevent self-comparisons. Likewise, all of the entries in the exploratory-to-exploratory comparison quadrant can be filled or otherwise blocked. Because these settings have not been tested, there is little value comparing them directly.

An empty entry is then selected at random. For example, the entry [2,5] can be selected. The next step depends on which quadrant the selected entry is located in. If the selected entry corresponds to a comparison between an exploitation and an exploration setting, then the mirror entry is also filled. In this example, the entry at [5,2] would also be filled. If the selected entry is in the exploitation only quadrant, then the mirror entry is not filled. This ensures that all sequences of exploitation settings are evaluated.

When the between-hub comparisons are repeated twice (i.e., the exploitation-to-exploitation settings), the hub-and-spoke produces a sequence with a length of

$$\frac{{n}^{2} - {m}^{2} - {( n - m )}^{2}}{2} + 2 m + 1 ,$$

which is 16 in the case of n=6 settings and m=3 hub settings. If the number of days is greater than the produced sequence length, comparisons among non-hub settings can be randomly generated to make up for the remaining days.

When the table, array, or matrix is filled, then a setting is selected at random from the row of current settings. If the row is full, one of the open settings is randomly selected. This process is repeated until all of the entries are filled.

An example sequence table, or array or matrix, that can be generated in this manner is illustrated in Table 4 below, and pseudo-code for the sequencing algorithm is shown in Table 5. In this example, the ordered sequence of control parameter settings was {2,5,3,2,4,3,1,4,2,3,6,1,3,1,2,6,2,1,5,1}.

next

| Current | 1 | 2 | 3 | 4 | 5 | 6 |
|---|---|---|---|---|---|---|
| 1 | X | 14 | 12 | 7 | 15 | 11 |
| 2 | 13 | X | 9 | 4 | 1 | 6 |
| 3 | 6 | 3 | X | 5 | 2 | 10 |
| 4 | 7 | 4 | 5 | X | X | X |
| 5 | 15 | 1 | 2 | X | X | X |
| 6 | 11 | 6 | 10 | X | X | X |

Pseudo-code of the Hub-and-Spoke sequencing algorithm

```
Inputs: number of known settings k_{K}; number of unknown settings kᵤ; total number of
settings: k = k_{K} + kᵤ; Sequence length: N;
Outputs: Sequence of settings S, where the value, Sₖ, represents the setting to be tested on
day k.
Initialization: A matrix M is generated to keep track of setting comparisons that have been
    tested. M = − [symbol]   ^{kxk}, where [symbol]    ^{kxk} is the identity matrix of size kxk; Each cell of matrix M
    is identified by its row and column, Cᵢ = [rᵢ, cᵢ]. The entire block of M corresponding to
    comparisons between unknown settings are also set to −1: M[kₖ + 1:k,:kₖ + 1:k] =
    −1; To initialize, a row is selected at random to be the first setting fort testing, r₁
Sampling:
for i = 1: N
  Select Cᵢ[rᵢ, cᵢ]:
      if (k > 1) The row, representing the current setting, is determined by the next setting
        from the previous selection, rᵢ = cᵢ₋₁;
      The next setting, cᵢ, is selected at random from the cells within row rᵢ whose values
        are zero.
      If there are no zero values in the row, then a random cell with zero value in the array
        and rᵢ andcᵢ will be selected.
      Mark cell Cᵢ[rᵢ, cᵢ] = i
      if (cᵢ or rᵢ > k_{K}) Mark cell Cᵢ^{′}[cᵢ, rᵢ] = i, so that comparisons between known and
        unknown are not repeated, but in reverse order.
      If there are no zeros left in row rᵢ then select an empty cell at random and use the
        two values as the next two in the sequence.
  Add row value to the sequence, Sₖ = rₖ
S_{N} = c_{N-1}
```

In this hub-and-spoke sequencing algorithm, the settings to be tested are arranged in a sequence to maximize the information obtained from the pairwise comparisons, resulting in a “calendar” that guides program usage. As a non-limiting example, a “hub-and-spoke” algorithm can be implemented for the sequencing algorithm. In general, such a hub-and-spoke algorithm produces a sequence that only compares the top m settings (i.e., hub settings with the highest predicted preference values) to the rest and to each other. The produced sequence maximizes the information that confirms if the high preference value still holds for the hubs and quickly spots new settings that win over the hubs. The hub-and-spoke algorithm also eliminates repeated comparison between hub and non-hub settings, but allows for repeated comparison between hubs.

It will be appreciated by those skilled in the art that the sequencing algorithms described above can also be used to order control parameter settings obtained, selected, or otherwise generated using techniques or algorithms other than the Bayesian preference model-based approach described in the present disclosure. In general, these sequencing algorithms can be used to create ordered sequences of control parameter settings, regardless of how those settings were generated or otherwise obtained, for the purposes of optimizing the order in which a user can test the control parameter settings.

The determined control parameter settings and ordered sequence in which the parameters should be tested by the user are then stored in a memory or other data storage device or medium, as indicated at step 108. For instance, the control parameter settings can be stored in a memory or data storage device or medium that is part of the medical device, or can be stored in memory or data storage device or medium that is remote to the medical device. In these latter instances, the sequence of control parameter settings can be stored for later use, at which time the sequence of control parameters can be communicated or otherwise transmitted or transferred to the medical device.

The control parameters can be used to control the medical device, as indicated at step 110. For instance, the control parameters can be retrieved, or otherwise acted upon, from the memory or other data storage device or medium, whether local or remote to the medical device. As described, the user can then test the control parameter settings (e.g., over a period of days to weeks) to generate user preference data that can be used to update future control parameter settings for the medical device, to validate the accuracy and reliability of the Bayesian preference model, or combinations thereof.

As mentioned above, the user's preference settings can also be validated. Examples of validating the user's preference settings are described below in more detail. In general, validation includes using the constructed Bayesian preference model to predict the user/patient preference. These predicted outcome data are then compared with the user's actual preferences, which may be collected through pairwise comparisons of the control parameter settings. If the user's preferences differ from the predicted outcomes, then the control parameter settings can optionally be updated, whether by resampling the Bayesian preference model (e.g., using a range of settings), by constructing a new Bayesian preference model based on the user preference data, or a combination of both.

In some instances the Bayesian preference model that is used to generate the control parameter settings can be validated with patient/user preference data to determine if the model is accurate and reliable. In general, validation protocols and metrics can be used to develop the credibility of these learned preference models. As one example, an internal validation method (e.g., k-fold validation) can be used, or a prospective validation (e.g., out-of-sample validation) can be used to test the model's prediction power on patient preference. As still another example, a clinical outcome validation method can be used, in which independently measured metrics of therapeutic outcomes are used.

In general, the validation protocols can be designed with at least two goals. One goal is to examine the prediction accuracy of the learned preference models, which can be achieved through internal validation and/or prospective validation studies. In both studies, models trained with a sub-dataset are used to predict the outcomes of the rest, and the model prediction accuracy can then be taken as the percentage of accurate predictions. In a non-limiting example, a second goal is to examine the relevance of the preference model's prediction to volitional movement in response to stimulation.

The predictive model can be used to calculate a probability of the observed outcome (binary), and a t-test on the accuracy, as compared to random shuffling of preference outcomes, can be performed to determine if the model's prediction is significantly better than chance. The probability predictions can in some instances be converted to metrics, such as Brier Skill Score (“BSS”) metrics, which can be easier to use to evaluate model quality. For example, if the BSS is greater than zero, then the model prediction is generally better than chance.

Preference scores produced by the model can also be fitted to objective efficacy metrics. The correlation can be used to show if the higher preference indicates higher efficacy in the aspect that the metric measures.

As noted, validation can include performing an internal validation. As a non-limiting example, internal validation can be conducted by dividing the data into N folds (e.g., N=5 folds), using N−1 folds to construct a Bayesian preference map that is used to predict the outcome of the final fold of data. The internal validation accuracy is then the average model prediction accuracy averaged across all folds. This can be repeated (e.g., N times), leaving one of the folds for testing out each time and training with the remaining N−1 folds. By averaging accuracy across the N folds, a final averaged accuracy can be reported.

In an example study, ten repeats of five-fold internal cross-validation was conducted on each participant's dataset to evaluate the prediction accuracy of the Bayesian preference model. For each time, the comparison data set collected for each participant were randomly divided into five even folds. Iteratively holding one-fold of data as the testing data set, and the other four folds as the training data set to assess the model prediction accuracy from the models fit to the training dataset. The average prediction accuracy across all folds and all repeats and its standard deviation was used to present the average model performance for each participant.

Noise is incorporated in the probit preference model to handle the inconsistencies in the observations and can be estimated through a hyperparameter optimization routine when fitting a preference Gaussian process model. Since the fitted preference value spectrum can vary individually, the SNR can be reported as a quantification of the inconsistencies found in each participant's data. The SNR for participant's Bayesian preference model can therefore be defined as:

$${SNR}_{i} = \frac{Range of Bayesian preference model}{Noise estimated in Bayesian preference model fit}$$

To further investigate the impact of SNR and compliance to the cross-validation accuracy, 5-fold cross-validation on 10 randomly subsampled datasets can be conducted.

Additionally or alternatively, validation can include performing a prospective validation. As a non-limiting example, prospective validation can be performed to show the prediction accuracy of the preference model while the patient is indicating their preference for the first time. This validation study protocol can utilize additional preference data, which can be collected at the end of each participant's temporal parameter preference learning phase. In the prospective validation study, the participant tests a number of different parameter settings in a row (e.g., nine or ten) and then tests the same settings again in a different order. The prospective validation accuracy is determined by comparing the predicted winner of each pairwise comparison in the two sequences, or by comparing how accurate the same comparisons are made, but using the two different sequences.

In an example study, prospective validation studies were performed on participants and the prediction accuracy of each participant's preference model in matching the observed outcomes was evaluated. Overall, the observed average prediction accuracy indicated that the preference model significantly helps to predict the correct preferred setting. Accuracy increases were noted for participants when considering the subset involving only the top number of settings (e.g., the top four settings). It is contemplated that this result was observed because the top settings are usually tested more times than the inferior settings when developing preference models and finding optimal stimulations. In general, the prospective validation study has statistically shown that the preference model learned through Bayesian preference optimization was representative of a participant's true preference.

Additionally or alternatively, validation can include performing a clinical outcome validation. As a non-limiting example, where the medical device is an electrical stimulation device for providing spinal cord stimulation, the clinical outcome validation can be performed to examine the correspondence between an objective measure of a participant's volitional movement response and the preference model prediction for the same stimulation parameters. For instance, the volitional movement measurement can be extracted from a daily triple flexion and extension task performed by the participants. Significant correlation between patient preference and clinical outcomes can be used to validate the Bayesian preference model.

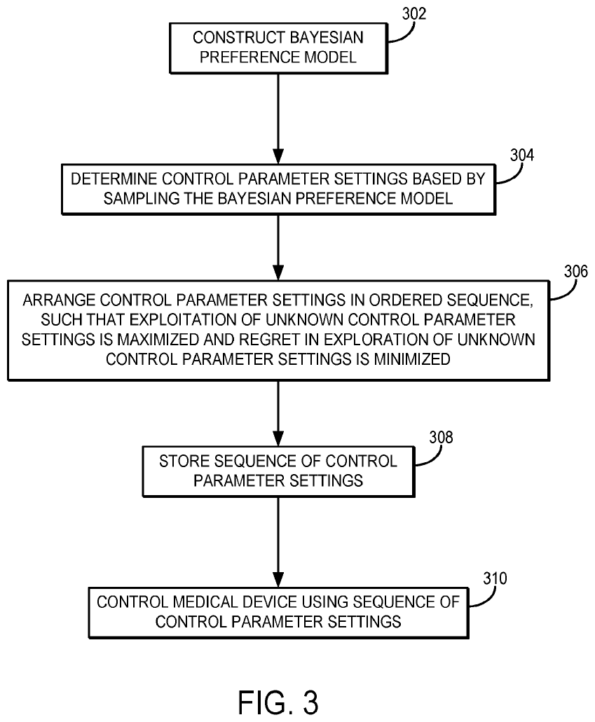

In some implementations, the method described above with respect to FIG. 1 can be adapted to include a process in which the control parameter settings are arranged in the ordered sequence such that exploitation of known control parameter settings is maximized and regret in exploration of unknown control parameter settings is minimized, as described above in more detail. In these instances, the method can be adapted as illustrated in the flowchart shown in FIG. 3, where step 106 from the method of FIG. 1 is adapted to include arranging the control parameter settings in the ordered sequence in the specified manner.

As shown in FIG. 3, the method includes constructing a Bayesian preference model at step 302, similar to the method described above with respect to step 102: determining control parameter settings based on sampling the Bayesian preference model at step 304, similar to the method described above with respect to step 104: arranging the control parameter settings in an ordered sequence at step 306: storing the sequence of control parameter settings at step 308, similar to the method described above with respect to step 108; and controlling a medical device using the sequence of control parameter settings at step 310, similar to the method described above with respect to step 110.

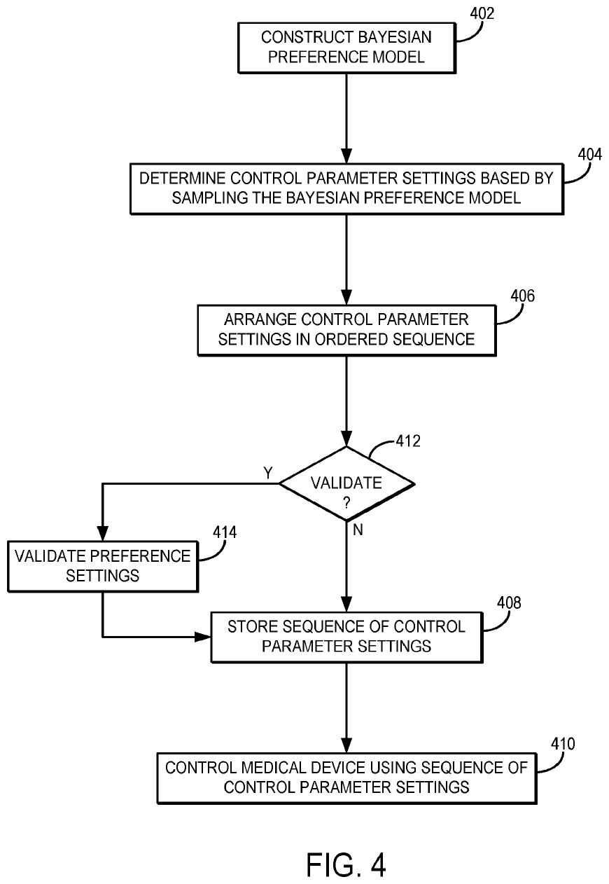

In some implementations, the method described above with respect to FIG. 1 can be adapted to include a process in which preference settings are validated before being stored for later use, as described above in more detail. In these instances, the method can be adapted as illustrated in the flowchart shown in FIG. 4 by adding a validation process, such as those described above in more detail.

As shown in FIG. 4, the method includes constructing a Bayesian preference model at step 402, similar to the method described above with respect to step 102: determining control parameter settings based on sampling the Bayesian preference model at step 404, similar to the method described above with respect to step 104: arranging the control parameter settings in an ordered sequence at step 406, similar to the method described above with respect to step 106: storing the sequence of control parameter settings at step 408, similar to the method described above with respect to step 108; and controlling a medical device using the sequence of control parameter settings at step 410, similar to the method described above with respect to step 110.

In this implementation, a validation check is made at decision block 412 after the control parameter settings have been arranged in the ordered sequence in step 406. If validation is selected, then the preference settings are validated at step 414 before they are stored at step 408. As described above, the validation process can use various different validation protocols, including internal validation, prospective validation, clinical outcome validation, combinations thereof, and so on.

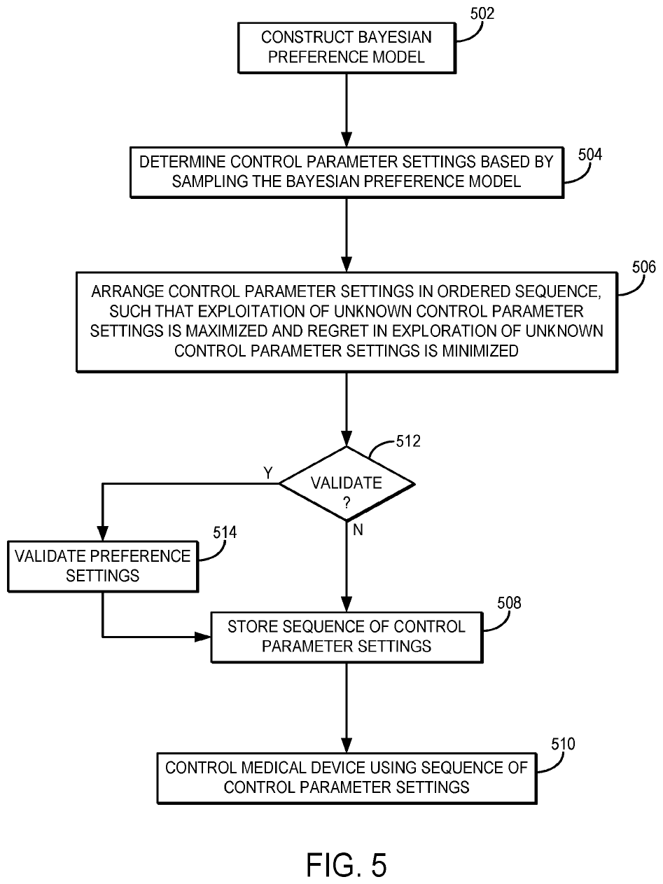

In some implementations, the method described above with respect to FIG. 1 can be adapted to include a process in which the control parameter settings are arranged in the ordered sequence such that exploitation of known control parameter settings is maximized and regret in exploration of unknown control parameter settings is minimized, as described above in more detail, and to include a validation of the preference settings, as also described above in detail. In these instances, the method can be adapted as illustrated in the flowchart shown in FIG. 5 by adapting step 106 from the method of FIG. 1 to include arranging the control parameter settings in the ordered sequence in the specified manner and by adding a validation process, such as those described above in more detail.

As shown in FIG. 5, the method includes constructing a Bayesian preference model at step 502, similar to the method described above with respect to step 102: determining control parameter settings based on sampling the Bayesian preference model at step 504, similar to the method described above with respect to step 104: arranging the control parameter settings in an ordered sequence at step 506; storing the sequence of control parameter settings at step 508, similar to the method described above with respect to step 108; and controlling a medical device using the sequence of control parameter settings at step 510, similar to the method described above with respect to step 110.

In this implementation, a validation check is made at decision block 512 after the control parameter settings have been arranged in the ordered sequence in step 506. If validation is selected, then the preference settings are validated at step 514 before they are stored at step 508. As described above, the validation process can use various different validation protocols, including internal validation, prospective validation, clinical outcome validation, combinations thereof, and so on.

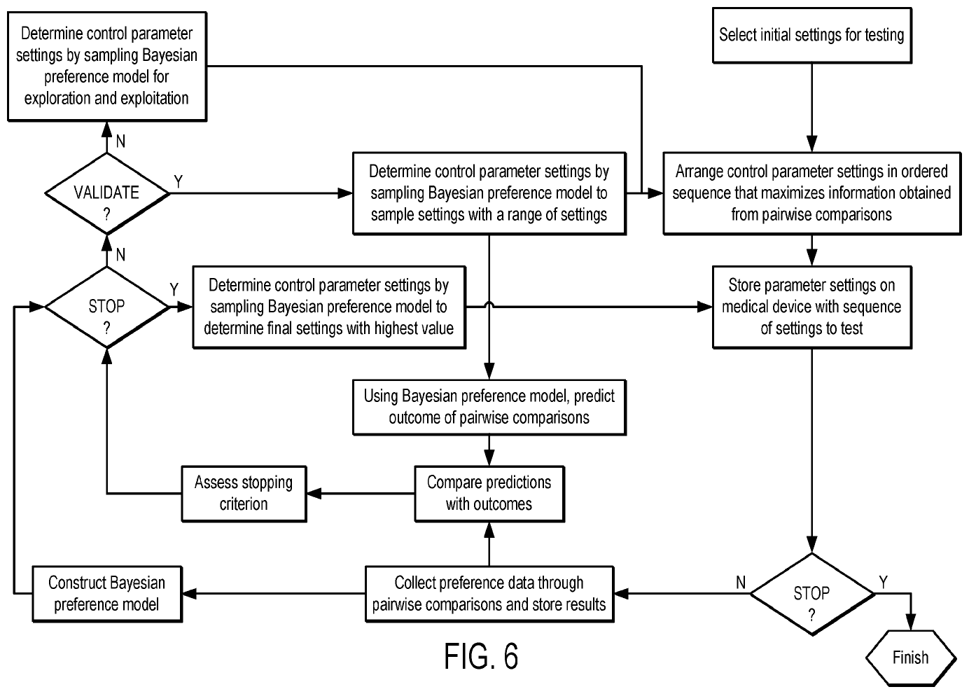

Referring now to FIG. 6, an example workflow for implementing a method for generating optimized control parameter settings is shown. Initial control parameter settings are selected for testing, or in some instances can be determined by sampling previously constructed Bayesian preference model. Control parameter settings can be determined by sampling a Bayesian preference model for exploration and exploitation. Additionally or alternatively, control parameter settings can be determined by sampling a Bayesian preference model to sample settings with a range of settings.

The control parameter settings are arranged in an ordered sequence, such as an ordered sequence that maximizes information obtained from pairwise comparisons between the control parameter settings. These control parameter settings can then be stored on a medical device together with the sequence of settings to test.

Preference data can be collected from a user through pairwise comparisons of the control parameter settings in the sequence, and the results of these comparisons stored. In some instances, these preference data can be used to compare predictions with outcomes, if available, which can be used to assess a stopping criterion for whether the control parameter settings should be updated. In other instances, the preference data can be used to construct, or otherwise update, a Bayesian preference model, as described above.

If a stopping criterion is satisfied the Bayesian preference model can be used to determine updated control parameter settings by sampling the Bayesian preference model to determine updated settings with the highest value. These updated control parameter settings can then be stored on the medical device for use by the patient.

If the stopping criterion is not satisfied, a decision can be made whether or not to perform validation. If so, then validation is performed and updated control parameter settings are determined by sampling the Bayesian preference model to sample settings with a range of settings. If validation is not performed, then updated control parameter settings can be determined by sampling the Bayesian preference model for exploration and exploitation. Using the Bayesian preference model, outcomes of pairwise comparisons can be predicted, which can be used when comparing predictions with outcomes.

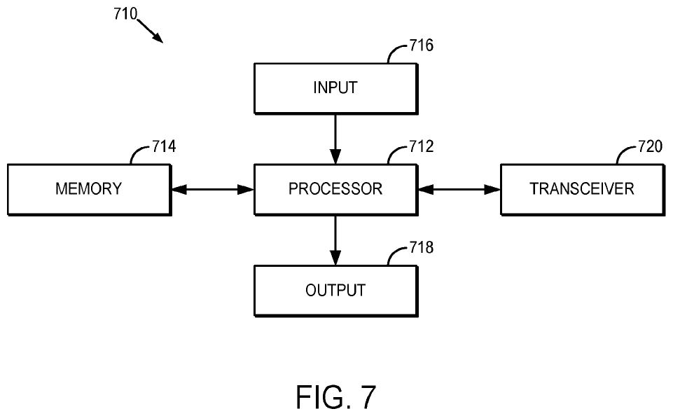

Referring now to FIG. 7, an example of a controller 710 that can implement the methods described in the present disclosure to control a controllable medical device is illustrated. In general, the controller 710 includes a processor 712, a memory 714, and input 716, and an output 718. The controller 710 can be implemented as part of a controllable medical device, or as a separate controller that is in communication with the controllable medical device via the output 718. As one example, the controller 710 can be implemented in a controllable medical device, such as an implantable medical device (e.g., an implanted nerve stimulation system or an implanted cardiac rhythm management system), a hearing aid, and so on. In other examples, the controller 710 can be implemented in a remote computer that communicates with the controllable medical device. In still other example, the controller 710 can be implemented in a smartphone that is paired with the controllable medical device, such as via Bluetooth or another wireless or wired communication.

In some embodiments, the input 716 is capable of sensing feedback data from the user. As one example, the feedback data can be electrophysiological activity, and the input 716 can be one or more electrodes. As another example, the feedback data can be chemical signal data, such as measured levels of chemicals. In such instances, the input 716 can include a suitable sensor for measuring the chemical signal data. As noted above, such a sensor could be a part of the controllable medical device, or could be a separate sensor that is in communication with the controller 710 via the input 716, whether through a wired or wireless connection. Such chemical data could also be measured through other means, such as via a blood sample taken from the user, and transmitted to the controller 710 via the input 716. The input 716 can thus more generally include a wired or wireless connector for receiving feedback data, which as noted above may also include behavioral or user preference data. In these latter examples, the feedback data can include a response surface generated from the behavioral or user preference data, and pairwise comparisons can be used to estimate values using, for example, a probit function. These feedback data can be transmitted to the controller 710 via the input 716.

The processor 712 includes at least one hardware processor to execute instructions embedded in or otherwise stored on the memory 714 to implement the methods described in the present disclosure. The memory can also store measured feedback data for processing, as well as settings to be provided to the processor 712 for generating control signals to be provided to a controllable medical device via the output 718. As described above, these settings can be stored and also updated by the adaptive control implemented by the controller 710.

The output 718 communicates control signals to a controllable medical device. As one example, where the controllable medical device is an electrical stimulation device, the control signals provided to the output 718 can control one or more electrodes to operate under control of the controller 710 to sense electrophysiological activity in a subject and to deliver electrical stimulations to the subject in response thereto. Sensing circuitry in the controller 710 can detect and processes electrophysiological activity sensed by the one or more one electrodes via the input 716 to determine the optimized stimulation settings (e.g., frequency, pulse width) based on the methods and algorithms described above. The optimized settings are provided as instructions to a pulse generator in the electrical stimulation device via the output 718, which in response to the instructions provides an electrical signal to the one or more electrodes to deliver the electrical stimulations to the subject.

The controller 710 can also include a transceiver 720 and associated circuitry for communicating with a programmer or other external or internal device. As one example, the transceiver 720 can include a telemetry coil. In some embodiments, the transceiver 720 can be a part of the input 716.

In operation, the controller 710 receives feedback data from the subject via the input 716. These feedback data are provided to the processor 712 where they are processed. For example, the processor 712 analyzes the feedback data and generates an appropriate response surface, or otherwise generates a Bayesian preference model used in the optimization to update the control parameter settings for the controllable medical device.

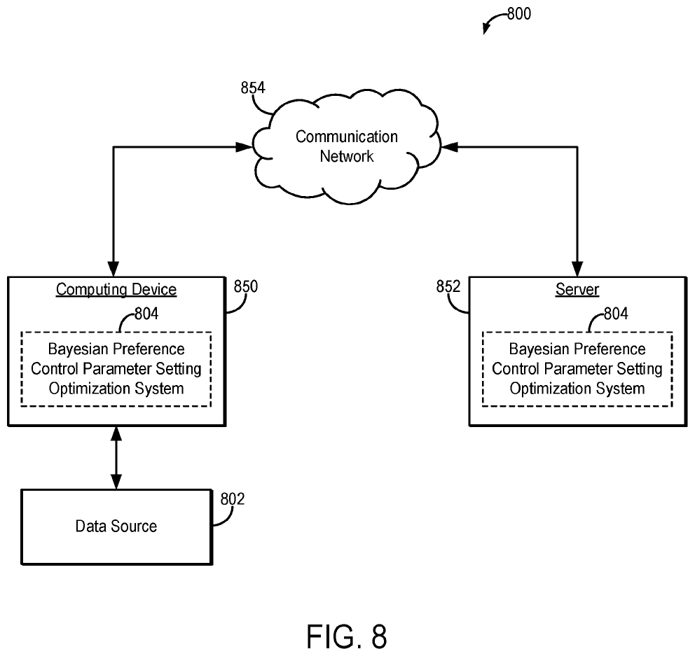

Referring now to FIG. 8, an example of a system 800 for determining or otherwise generating an optimized sequence of control parameter settings in accordance with some embodiments of the systems and methods described in the present disclosure is shown. As shown in FIG. 8, a computing device 850 can receive one or more types of data (e.g., user preference data, other feedback data) from data source 802. In some embodiments, computing device 850 can execute at least a portion of a Bayesian preference control parameter setting optimization system 804 to generate an optimized sequence of control parameter settings for a medical device from data received from the data source 802.

Additionally or alternatively, in some embodiments, the computing device 850 can communicate information about data received from the data source 802 to a server 852 over a communication network 854, which can execute at least a portion of the Bayesian preference control parameter setting optimization system 804. In such embodiments, the server 852 can return information to the computing device 850 (and/or any other suitable computing device) indicative of an output of the Bayesian preference control parameter setting optimization system 804.

In some embodiments, computing device 850 and/or server 852 can be any suitable computing device or combination of devices, such as a desktop computer, a laptop computer, a smartphone, a tablet computer, a wearable computer, a server computer, a virtual machine being executed by a physical computing device, and so on. The computing device 850 and/or server 852 can also generate an optimized sequence of control parameter settings from the data and/or validate a Bayesian preference model or sequence of control parameter settings.

In some embodiments, data source 802 can be any suitable source of data (e.g., user preference data, other feedback data). In some embodiments, data source 802 can be local to computing device 850. For example, data source 802 can be incorporated with computing device 850 (e.g., computing device 850 can be configured as part of a device for capturing, scanning, and/or storing data). As another example, data source 802 can be connected to computing device 850 by a cable, a direct wireless link, and so on. Additionally or alternatively, in some embodiments, data source 802 can be located locally and/or remotely from computing device 850, and can communicate data to computing device 850 (and/or server 852) via a communication network (e.g., communication network 854).

In some embodiments, communication network 854 can be any suitable communication network or combination of communication networks. For example, communication network 854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 854 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, proprietary or dedicated secure medical device interfaces, and so on.

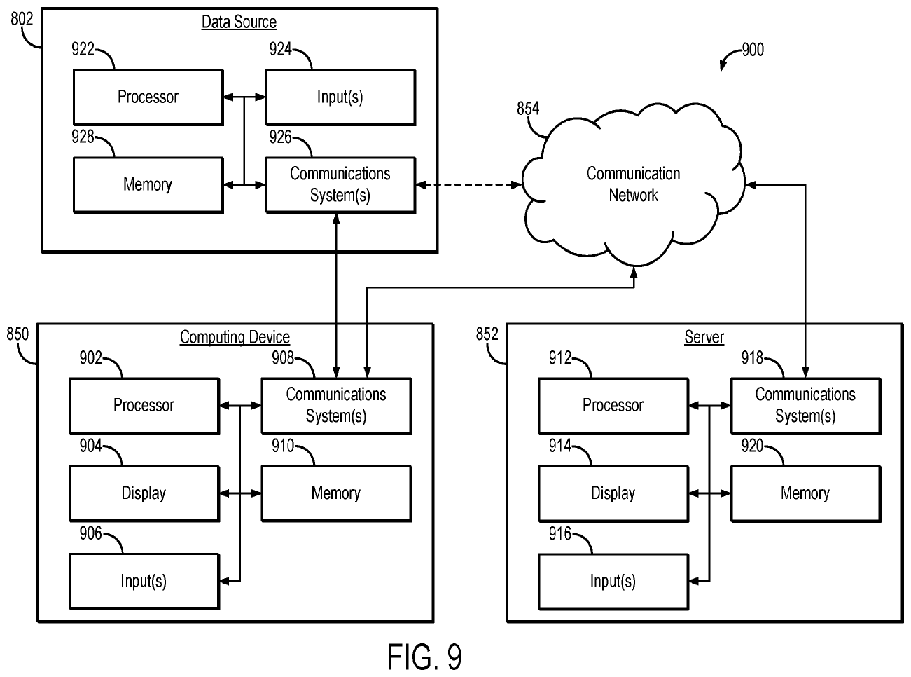

Referring now to FIG. 9, an example of hardware 900 that can be used to implement data source 802, computing device 850, and server 852 in accordance with some embodiments of the systems and methods described in the present disclosure is shown. As shown in FIG. 9, in some embodiments, computing device 850 can include a processor 902, a display 904, one or more inputs 906, one or more communication systems 908, and/or memory 910. In some embodiments, processor 902 can be any suitable hardware processor or combination of processors, such as a central processing unit (“CPU”), a graphics processing unit (“GPU”), and so on. In some embodiments, display 904 can include any suitable display devices, such as a computer monitor, a touchscreen, a television, and so on. In some embodiments, inputs 906 can include any suitable input devices and/or sensors that can be used to receive user input, such as a keyboard, a mouse, a touchscreen, a microphone, and so on.

In some embodiments, communications systems 908 can include any suitable hardware, firmware, and/or software for communicating information over communication network 854 and/or any other suitable communication networks. For example, communications systems 908 can include one or more transceivers, one or more communication chips and/or chip sets, and so on. In a more particular example, communications systems 908 can include hardware, firmware and/or software that can be used to establish a Wi-Fi connection, a Bluetooth connection, a cellular connection, an Ethernet connection, and so on.

In some embodiments, memory 910 can include any suitable storage device or devices that can be used to store instructions, values, data, or the like, that can be used, for example, by processor 902 to present content using display 904, to communicate with server 852 via communications system(s) 908, and so on. Memory 910 can include any suitable volatile memory, non-volatile memory, storage, or any suitable combination thereof. For example, memory 910 can include RAM, ROM, EEPROM, one or more flash drives, one or more hard disks, one or more solid state drives, one or more optical drives, and so on. In some embodiments, memory 910 can have encoded thereon, or otherwise stored therein, a computer program for controlling operation of computing device 850. In such embodiments, processor 902 can execute at least a portion of the computer program to present content (e.g., user interfaces, graphics, tables), receive content from server 852, transmit information to server 852, and so on. For example, the processor 902 and the memory 910 can be configured to perform the methods described herein (e.g., the method of FIG. 1, the method of FIG. 3, the method of FIG. 4, the method of FIG. 5, the method of FIG. 6).

In some embodiments, server 852 can include a processor 912, a display 914, one or more inputs 916, one or more communications systems 918, and/or memory 920. In some embodiments, processor 912 can be any suitable hardware processor or combination of processors, such as a CPU, a GPU, and so on. In some embodiments, display 914 can include any suitable display devices, such as a computer monitor, a touchscreen, a television, and so on. In some embodiments, inputs 916 can include any suitable input devices and/or sensors that can be used to receive user input, such as a keyboard, a mouse, a touchscreen, a microphone, and so on.

In some embodiments, communications systems 918 can include any suitable hardware, firmware, and/or software for communicating information over communication network 854 and/or any other suitable communication networks. For example, communications systems 918 can include one or more transceivers, one or more communication chips and/or chip sets, and so on. In a more particular example, communications systems 918 can include hardware, firmware and/or software that can be used to establish a Wi-Fi connection, a Bluetooth connection, a cellular connection, an Ethernet connection, and so on.

In some embodiments, memory 920 can include any suitable storage device or devices that can be used to store instructions, values, data, or the like, that can be used, for example, by processor 912 to present content using display 914, to communicate with one or more computing devices 850, and so on. Memory 920 can include any suitable volatile memory, non-volatile memory, storage, or any suitable combination thereof. For example, memory 920 can include RAM, ROM, EEPROM, one or more flash drives, one or more hard disks, one or more solid state drives, one or more optical drives, and so on. In some embodiments, memory 920 can have encoded thereon a server program for controlling operation of server 852. In such embodiments, processor 912 can execute at least a portion of the server program to transmit information and/or content (e.g., data, a user interface) to one or more computing devices 850, receive information and/or content from one or more computing devices 850, receive instructions from one or more devices (e.g., a personal computer, a laptop computer, a tablet computer, a smartphone), and so on.

In some embodiments, the server 852 is configured to perform the methods described in the present disclosure. For example, the processor 912 and memory 920 can be configured to perform the methods described herein (e.g., the method of FIG. 1, the method of FIG. 3, the method of FIG. 4, the method of FIG. 5, the method of FIG. 6).

In some embodiments, data source 802 can include a processor 922, one or more inputs 924, one or more communications systems 926, and/or memory 928. In some embodiments, processor 922 can be any suitable hardware processor or combination of processors, such as a CPU, a GPU, and so on. In some embodiments, the one or more inputs 924 are generally configured to acquire data. Additionally or alternatively, in some embodiments, one or more inputs 924 can include any suitable hardware, firmware, and/or software for coupling to and/or controlling operations of a medical device. In some embodiments, one or more portions of the one or more inputs 924 can be removable and/or replaceable.

Note that, although not shown, data source 802 can include any suitable inputs and/or outputs. For example, data source 802 can include input devices and/or sensors that can be used to receive user input, such as a keyboard, a mouse, a touchscreen, a microphone, a trackpad, a trackball, a smartphone, a tablet, and so on. As another example, data source 802 can include any suitable display devices, such as a computer monitor, a touchscreen, a television, a smartphone, a tablet, etc., one or more speakers, and so on.

In some embodiments, communications systems 926 can include any suitable hardware, firmware, and/or software for communicating information to computing device 850 (and, in some embodiments, over communication network 854 and/or any other suitable communication networks). For example, communications systems 926 can include one or more transceivers, one or more communication chips and/or chip sets, and so on. In a more particular example, communications systems 926 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, a proprietary or dedicated secure medical device interface, and so on.

In some embodiments, memory 928 can include any suitable storage device or devices that can be used to store instructions, values, data, or the like, that can be used, for example, by processor 922 to control the one or more inputs 924, and/or receive data from the one or more inputs 924; to generate a sequence of control parameter settings from data: present content (e.g., a user interface) using a display: communicate with one or more computing devices 850; and so on. Memory 928 can include any suitable volatile memory, non-volatile memory, storage, or any suitable combination thereof. For example, memory 928 can include RAM, ROM, EEPROM, one or more flash drives, one or more hard disks, one or more solid state drives, one or more optical drives, and so on. In some embodiments, memory 928 can have encoded thereon, or otherwise stored therein, a program for controlling operation of data source 802. In such embodiments, processor 922 can execute at least a portion of the program to generate control parameter settings, transmit information and/or content (e.g., data) to one or more computing devices 850, receive information and/or content from one or more computing devices 850, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A controller for controlling a controllable medical device, comprising:
an input that receives feedback data representative of a treatment response or effect in a subject;
a processor in communication with the input and programmed to:
receive the feedback data from the input and generate a Bayesian preference model therefrom;
generate control parameter settings by sampling the Bayesian preference model;
arrange the control parameter settings in an ordered sequence for testing the control parameter settings, wherein the processor is programmed to arrange the control parameter settings in the ordered sequence such that exploitation of known control parameter settings is maximized and regret in exploration of unknown control parameter settings is minimized:
a memory in communication with the input and the processor, wherein the memory stores instructions for generating control parameter settings, the feedback data received from the input, and the ordered sequence of control parameter settings generated by the processor; and
an output that communicates the ordered sequence of control parameter settings to a controllable medical device.

2. The controller as recited in claim 1, wherein the processor is further programmed to validate the Bayesian preference model according to a validation protocol by at least one of predicting subject preference outcomes of a sequence of comparisons using the Bayesian preference model or programming the controller with the ordered sequence of control parameter settings and comparing the predicted outcome to a subject preference outcome.

3. A controller for controlling a controllable medical device, comprising:
an input that receives feedback data representative of a treatment response or effect in a subject;
a processor in communication with the input and programmed to:
receive the feedback data from the input and generate a Bayesian preference model therefrom;
generate control parameter settings by sampling the Bayesian preference model;
arrange the control parameter settings in an ordered sequence for testing the control parameter settings;
validate the Bayesian preference model according to a validation protocol by at least one of:
predicting subject preference outcomes of a sequence of comparisons using the Bayesian preference model; or
programming the controller with the ordered sequence of control parameter settings and comparing the predicted outcome to a subject preference outcome;
a memory in communication with the input and the processor, wherein the memory stores instructions for generating control parameter settings, the feedback data received from the input, and the ordered sequence of control parameter settings generated by the processor; and
an output that communicates the ordered sequence of control parameter settings to a controllable medical device.

4. The controller as recited in claim 3, wherein the processor is configured to arrange the control parameter settings in the ordered sequence such that exploitation of known control parameter settings is maximized and regret in exploration of unknown control parameter settings is minimized.

5. The controller as recited in any one of claim 1 or 3, wherein the feedback data received from the input comprise at least one of behavior metrics or user preferences.

6. The controller as recited in claim 5, wherein the processor is programmed to generate a probit function based on the feedback data.

7. The controller as recited in claim 6, wherein the feedback data comprise user preferences between two different control parameter settings.

8. The controller as recited in any one of claim 1 or 3, wherein the processor is configured to arrange the control parameter settings in the ordered sequence such that information obtained from pairwise comparison of the control parameter settings is maximized.

9. The controller as recited in any one of claim 1 or 3, wherein the processor is configured to arrange the control parameter settings in the ordered sequence such that information about subject preference to different control parameter settings is maximized.

10. The controller as recited in any one of claim 1 or 3, wherein the processor is programmed to generate the control parameter settings by sampling the Bayesian preference model using batch sampling.

11. The controller as recited in any one of claim 2 or 3, wherein the validation protocol is an internal validation protocol comprising a k-fold validation.

12. The controller as recited in any one of claim 2 or 3, wherein the validation protocol is a prospective validation protocol comprising an out-of-sample validation.

13. A controller for controlling a controllable medical device, comprising:
an input configured to receive feedback data representative of a treatment response or effect in a subject;
a memory, wherein the memory stores the feedback data received from the input and control parameter settings for controlling a controllable medical device;
a processor in communication with the input and the memory, the processor being programmed to:
receive the feedback data from the input;
receive control parameter settings from the memory; and
arrange the control parameter settings in an ordered sequence for testing the control parameter settings by the subject based at least in part on the feedback data; and
an output that communicates the ordered sequence of control parameter settings to a controllable medical device.

14. The controller as recited in claim 13, wherein the processor is configured to arrange the control parameter settings in the ordered sequence such that information in the feedback data obtained from pairwise comparison of the control parameter settings is maximized.

15. The controller as recited in claim 13, wherein the processor is configured to arrange the control parameter settings in the ordered sequence such that information from the feedback data about subject preference to different control parameter settings is maximized.

16. The controller as recited in claim 13, wherein the processor is configured to arrange the control parameter settings in the ordered sequence such that exploitation of known control parameter settings is maximized and regret in exploration of unknown control parameter settings is minimized.
